(19) United States
(12) Patent Application Publication
(10) Pub. No.: US 2017/0181857 A1
(43) Pub. Date: Jun. 29, 2017

(54) ACETABULAR CUP STRUCTURE

(71) Applicant: METAL INDUSTRIES RESEARCH & DEVELOPMENT CENTRE, Kaohsiung (TW)

(72) Inventors: Wei-Ching WANG, Kaohsiung City (TW); Yih-Wen TARNG, Kaohsiung City (TW); Bing-Feng HUANG, Kaohsiung CIty (TW); Chia-Min WEI, Kaohsiung City (TW); Chun-Chieh WANG, Kaohsiung City (TW)

(73) Assignee: Metal Industries Research & Development Centre

(21) Appl. No.: 15/216,339

(22) Filed: Jul. 21, 2016

(30) Foreign Application Priority Data
Dec. 29, 2015 (TW) 104144220

(51) Int. Cl. A61F 2/34; A61F 2/36

(52) U.S. Cl. CPC A61F 2/34; A61F 2/36; A61F 2002/30649

(57) ABSTRACT

An acetabular cup structure of the present invention includes a ball-and-socket prosthesis and a liner, where a ball wall of the ball-and-socket prosthesis has a groove formed by connecting at least two continuous straight lines, and the groove connects an outer surface of the ball wall to an inner surface of the ball wall, so that an expandable sheet is formed between the groove formed by connecting the at least two continuous straight lines and having an angle there-between and two ends of the groove that are not connected, and the liner, for connecting a femoral stem, is tightly attached to the inner surface of the ball wall, and when the liner is tightly attached to the inner surface of the ball wall, the liner presses the sheet and extends to the groove, so that the liner is attached to the ball-and-socket prosthesis more tightly.

### CROSS-REFERENCE TO RELATED APPLICATION

This application claims the benefit of Taiwan Patent Application No. 104144220, filed on Dec. 29, 2015, which is hereby incorporated by reference for all purposes as if fully set forth herein.

### BACKGROUND

Technical Field

The present invention relates to an acetabular cup structure, and in particular, to an acetabular cup structure having an expandable sheet.

Related Art

The hip joint is the joint between the acetabulum and the femoral head. The hip joint, cartilage, synovial membrane and fibrous capsule form a ball and socket joint capable of motions in many directions. The structure of the hip joint is like a ball contained in a socket, which allows the hip joint to have enough stability to support the body weight. Currently, an acetabular cup for an artificial joint often loosens because of osteolysis around the acetabular cup caused by macrophages, so more surgeries must be performed to repair the acetabulum frequently. In another aspect, the cancellous bone of an aged patient cannot fix the acetabular cup in the usual fixing manner, and bone cement must be used, so the patient needs more time to recover from a surgery.

In the conventional art, for example, U.S. Pat. No. 4,715,859 A disclosed an acetabular cup that does not involve connection during implantation. Instead, flanks are directly anchored in the acetabular cup to support the femoral head. However, this design cannot withstand high stress generated during movements of the human body, and becomes loosened in short time, so the firmness of the design is not desirable.

In addition, in the conventional art, for example, ROC Patent Publication No. 515710 disclosed a composite acetabular cup for an artificial hip joint. A liner and a shell of the acetabular cup are made of a biocompatible metal or molded alloy, for example, a titanium alloy, a cobalt-chromium-molybdenum alloy, and stainless steel. An intermediate layer sandwiched between the liner and the shell is made of an elastic material such as polyethylene. A coated layer made of porous particles, metal meshes, hydroxyapatite, or bone cement may be coated on the outside of the acetabular cup, so as to join and fix the bone tissues of the acetabulum. Moreover, the fixing manner may even be screw fixation.

However, the foregoing conventional art did not particularly propose the design that the acetabular cup has an expandable sheet for more tightly fixing the liner on the ball-and-socket prosthesis.

### SUMMARY

An objective of the present invention is to provide an acetabular cup structure. By using an expandable sheet structure formed with a groove that connects an outer surface to an inner surface of a ball wall on the ball wall of the ball-and-socket prosthesis, when the liner is tightly attached to the ball-and-socket prosthesis, a part of the liner extends to the groove, so that the liner is attached to the ball-and-socket prosthesis more tightly.

The acetabular cup structure of the present invention includes a ball-and-socket prosthesis and a liner, where a ball wall of the ball-and-socket prosthesis has a groove formed by connecting at least two continuous straight lines, and the groove connects an outer surface of the ball wall to an inner surface of the ball wall, so that an expandable sheet is formed between the groove formed by connecting the at least two continuous straight lines and having an angle there-between and two ends of the groove that are not connected, and the liner, for connecting a femoral stem, is tightly attached to the inner surface of the ball wall, and when the liner is tightly attached to the inner surface of the ball wall, the liner presses the sheet and extends to the groove, so that the liner is attached to the ball-and-socket prosthesis more tightly.

### BRIEF DESCRIPTION OF THE DRAWINGS

The disclosure will become more fully understood from the detailed description given herein below for illustration only, and thus are not limitative of the disclosure, and wherein:

FIG. 1 is a schematic three-dimensional diagram of an acetabular cup structure according to the present invention;

FIG. 2 is a three-dimensional diagram of a ball-and-socket prosthesis of the acetabular cup structure according to the present invention;

FIG. 3 is a three-dimensional diagram illustrating combination of the ball-and-socket prosthesis and a liner of the acetabular cup structure according to the present invention;

FIGS. 4 and 5 are schematic diagrams of a 17 -shaped groove of the acetabular cup structure according to the present invention; and

FIGS. 6 and 7 are schematic diagrams of an arc groove of the acetabular cup structure according to the present invention.

### DETAILED DESCRIPTION

To make the foregoing and other objectives, features and advantages of the present invention more obvious, the present invention is described in the following in detail with reference to the accompanying drawings.

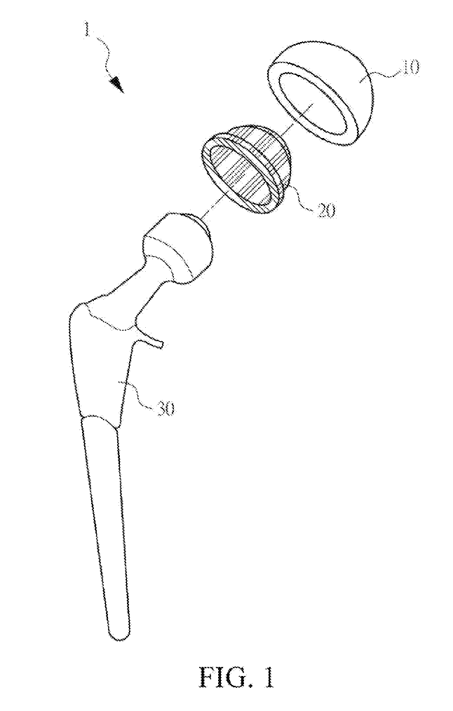

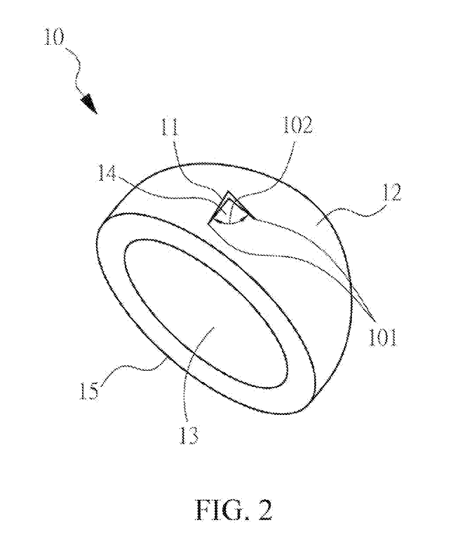

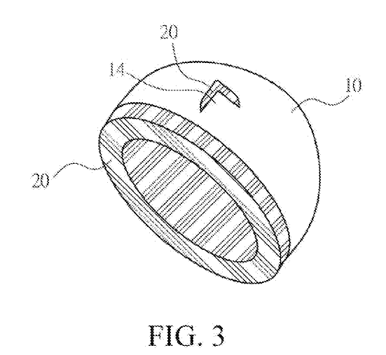

First, refer to FIGS. 1 to 3. FIG. 1 is a schematic three-dimensional diagram of an acetabular cup structure according to the present invention, FIG. 2 is a three-dimensional diagram of a ball-and-socket prosthesis of the acetabular cup structure according to the present invention, and FIG. 3 is a three-dimensional diagram illustrating combination of the ball-and-socket prosthesis and a liner of the acetabular cup structure according to the present invention. The acetabular cup structure 1 of the present invention includes a ball-and-socket prosthesis 10, where a ball wall of the ball-and-socket prosthesis 10 has a groove 11 formed by connecting at least two continuous straight lines, and the groove 11 connects an outer surface 12 of the ball wall to an inner surface 13 of the ball wall, so that an expandable sheet 14 is formed between the groove formed by connecting the two continuous straight lines and having an angle 102 there-between and two ends 101 of the groove that are not connected.

The ball-and-socket prosthesis is made of a memory metal, most preferably a memory titanium alloy, which is implanted into an acetabulum of a human body when the prosthesis is deformable at a low temperature (0° C.). When the temperature rises to the body temperature (about 35° C.), the ball-and-socket prosthesis returns to the shape of the acetabulum of the human body, and is effectively attached to the acetabulum of the human body tightly.

Then, a liner 20, for connecting a femoral stem 30, is tightly attached to the inner surface 13 of the ball wall. When the liner 20 is tightly attached to the inner surface 13 of the ball wall, the liner presses the sheet 14 and extends to the groove 11, so that the liner 20 is attached to the ball-and-socket prosthesis 10 more tightly. The liner is made of a high-molecular material or a ceramic material.

The femoral stem 30 is used for connecting a femoral head of the human body. As described above, a bottom edge 15 of the ball-and-socket prosthesis 10 is continuous, that is, the groove does not extend to the bottom edge to form an opening. Because the bottom edge 15 is continuous, the size of the bottom edge 15 is fixed and does not shrink, which can effectively prevent over expansion caused by marginal bone resorption or expansion of the sheet outside the prosthesis caused by the liner loosens. The bottom edge 15 matches the site of the bone defect, which prevents the problems of self-rotation and dislocation.

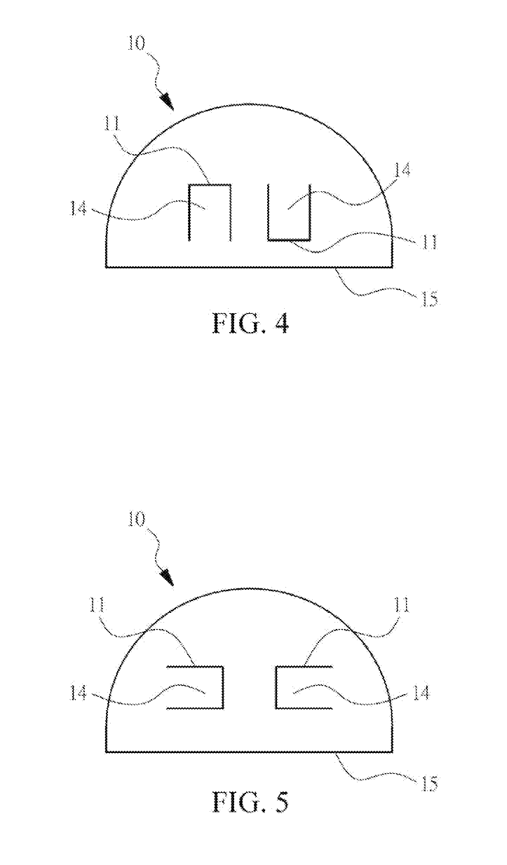

Next, refer to FIGS. 4 and 5. FIGS. 4 and 5 are schematic diagrams of a [symbol] -shaped groove of the acetabular cup structure according to the present invention. The groove may be a [symbol] shape formed by three continuous straight lines, and the formed sheet 14 is rectangular or square. In FIG. 4, the [symbol] -shaped groove is perpendicular to the bottom edge 15 of the ball-and-socket prosthesis, to form a [symbol] shape or an inverted [symbol] shape. The [symbol] -shaped or inverted [symbol] -shaped groove enables the liner to be attached to the ball-and-socket prosthesis more tightly to resist vertical stresses. Therefore, when the human jumps up and down, the dislocation of the liner can be prevented effectively.

Moreover, in FIG. 5, the [symbol] -shaped groove may be parallel to a bottom edge of the ball-and-socket, and forms a [symbol] shape or an inverted [symbol] shape. The [symbol] -shaped or inverted [symbol] -shaped groove enables the liner to be attached to the ball-and-socket prosthesis more tightly to resist stresses generated by rotation and twisting. Therefore, when the human twists, the dislocation of the liner can be prevented effectively.

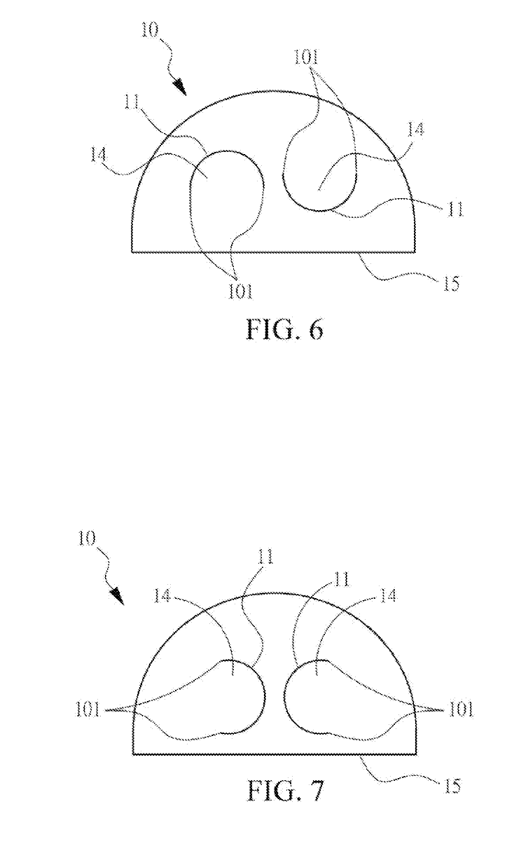

Next, refer to FIGS. 6 and 7. FIGS. 6 and 7 are schematic diagrams of an arc groove of the acetabular cup structure according to the present invention. The structure of the ball-and-socket prosthesis 10 is similar to that describe above, and the difference lies in that the ball wall of the ball-and-socket prosthesis 10 may have an arc groove 11, so that a sheet 14 is formed in a portion enclosed by the groove and two ends 101 of the groove.

In FIG. 6, the arc is downwards or upwards, and the groove of this design enables the liner to be attached to the ball-and-socket prosthesis more tightly to resist vertical stresses. In addition, the arc may be towards left or right, and the groove of this design enables the liner to be attached to the ball-and-socket prosthesis more tightly to resist stresses generated by rotation and twisting.

In the acetabular cup structure according to the present invention, by using an expandable sheet structure formed with a groove that connects an outer surface to an inner surface of a ball wall on the ball wall of the ball-and-socket prosthesis, when the liner is tightly attached to the ball-and-socket prosthesis, a part of the liner extends to the groove, so that the liner is attached to the ball-and-socket prosthesis more tightly.

Implementation manners or embodiments of technical means of the present invention for solving problems are described above, which are not intended to limit the scope of the present invention. All equivalent changes and modifications based on the claims of the present invention shall fall within the protection scope of the present invention.

What is claimed is:

1. An acetabular cup structure, comprising:
a ball-and-socket prosthesis, wherein a ball wall of the ball-and-socket prosthesis has a groove formed by connecting at least two continuous straight lines, and the groove connects an outer surface of the ball wall to an inner surface of the ball wall, so that an expandable sheet is formed between the groove formed by connecting the at least two continuous straight lines and having an angle there-between and two ends of the groove that are not connected, and
a liner, for connecting a femoral stem, and tightly attached to the inner surface of the ball wall,
wherein when the liner is tightly attached to the inner surface of the ball wall, the liner presses the sheet and extends to the groove, so that the liner is attached to the ball-and-socket prosthesis more tightly.

2. The acetabular cup structure according to claim 1, wherein a bottom edge of the ball-and-socket prosthesis is continuous.

3. The acetabular cup structure according to claim 1, wherein the groove has a [symbol] shape formed by three continuous straight lines, and the formed sheet is rectangular or square.

4. The acetabular cup structure according to claim 3, wherein the [symbol] -shaped groove is perpendicular to a bottom edge of the ball-and-socket prosthesis, and has a [symbol] -shaped form or an inverted [symbol] -shaped form.

5. The acetabular cup structure according to claim 3, wherein the [symbol] -shaped groove is parallel to a bottom edge of the ball-and-socket prosthesis, and has a [symbol] -shaped form or an inverted [symbol] -shaped form.

6. An acetabular cup structure, comprising:
a ball-and-socket prosthesis, wherein a ball wall of the ball-and-socket prosthesis has an arc groove, and the groove connects an outer surface of the ball wall to an inner surface of the ball wall, so that an expandable sheet is formed in a portion enclosed by the groove and two ends of the groove, and
a liner, for connecting a femoral stem, and tightly attached to the inner surface of the ball wall,
wherein when the liner is tightly attached to the inner surface of the ball wall, the liner presses the sheet and extends to the groove, so that the liner is attached to the ball-and-socket prosthesis more tightly.

7. The acetabular cup structure according to claim 6, wherein the arc is upwards or downwards.

8. The acetabular cup structure according to claim 6, wherein the arc is towards left or right.

9. The acetabular cup structure according to claim 6, wherein a bottom edge of the ball-and-socket prosthesis is continuous.
